(19) United States
(12) Patent Application Publication
(10) Pub. No.: US 2025/0012699 A1
(43) Pub. Date: Jan. 9, 2025

(54) METHOD OF DETERMINING EFFICIENCY OF INCREASING SOIL CARBON SINK BASED ON GREEN MANURE RETURNING TO FIELDS AND SYSTEM THEREOF

(71) Applicants: Hainan Institute of Zhejiang University, Sanya City (CN); Zhejiang University, Hangzhou City (CN); Hohai University, Nanjing City (CN)

(72) Inventors: Yong HE, Sanya City (CN); Peng WANG, Hangzhou City (CN); Qianjing JIANG, Hangzhou City (CN); Yawei LI, Nanjing City (CN); Junzeng XU, Nanjing City (CN)

(21) Appl. No.: 18/428,936

(22) Filed: Jan. 31, 2024

(30) Foreign Application Priority Data
Jul. 4, 2023 (CN) 202310811749.3

(51) Int. Cl. G01N 15/08; G01N 33/24

(52) U.S. Cl. CPC G01N 15/08; G01N 33/24; G01N 2015/0846; G01N 33/245

(57) ABSTRACT

The present disclosure discloses a method of determining efficiency of increasing a soil carbon sink based on green manure returning to fields and a system thereof. The method includes: determining green manure biomass of a target farmland based on an unmanned aerial vehicle lidar technology during a fallow period of the target farmland before green manure is turned over; calculating carbon biomass of the target farmland based on the green manure biomass; calculating microbial soil carbon pump efficiency and mineral carbon pump efficiency after the green manure of the target farmland is turned over; and determining a soil carbon sink effect of turning over green manure on the target farmland according to the carbon biomass, the microbial soil carbon pump efficiency and the mineral carbon pump efficiency. The present disclosure realizes the quantitative calculation of efficiency of increasing the soil carbon sink of green manure.

### CROSS-REFERENCE TO RELATED APPLICATION

This patent application claims the benefit and priority of Chinese Patent Application No. 2023108117493, filed with the China National Intellectual Property Administration on Jul. 4, 2023, the disclosure of which is incorporated by reference herein in its entirety as part of the present application.

### TECHNICAL FIELD

The present disclosure relates to the technical field of soil management, in particular to a method of determining efficiency of increasing a soil carbon sink based on green manure returning to fields and a system thereof.

### BACKGROUND

Excessive use of chemical fertilizers has led to the long-term sub-health of agricultural production land in many areas, the continuous deterioration of soil health and the continuous decline of soil fertility, which restricts the high and stable yield of grain. The low utilization rate of fertilizers is the main limiting factor of agricultural development. Relying solely on a huge amount of chemical fertilizer investment cannot sustainably increase grain production, but will instead have a serious impact on the sustainability of the ecological environment and people's lives and health.

Green Manure (GM), as a kind of bio-fertilizers with complete nutrients, can provide various mineral nutrients for crops after decomposition, which has the functions of increasing soil organic matters, improving the soil structure and improving the soil fertility. GM is a key measure to establish a good agricultural ecological environment and realize green and sustainable development of agriculture, and is of great significance to improve the crop yield and the crop nutrient utilization efficiency. China is particularly rich in green manure resources, in which 73% of the green manure kinds are classified as leguminous green manure, mainly including Chinese milk vetch, alfalfa and Vicia sativa. There are a large number of Rhizobia in the roots of this kind of green manure, and the annual nitrogen fixation capacity can be up to 110 to 227 kg*ha-1. Therefore, planting green manure in a fallow period and returning green manure which has been turned over to fields can effectively replace some chemical fertilizers, and promote the steady improvement of the soil carbon and nitrogen holding capacity and holding efficiency.

A paddy field is one of the main sources of non-CO₂ greenhouse gas emissions in agricultural production, and the CH₄ emission thereof accounts for about 20% of the total emissions. However, at the same time, the efficiency of increasing the paddy field soil carbon sink also plays an important role in the balance of inputting and outputting carbon and nitrogen in the production process. Therefore, the accurate evaluation of the paddy field soil carbon sink and the efficiency is of great significance for understanding the carbon sequestration potential of paddy field soil and achieving the goal of peak carbon dioxide emissions and carbon neutrality in the agriculture field.

Because of the differences in farming modes and management measures, the soil carbon density in different agricultural areas is quite different. Moreover, because of the differences in climatic conditions and management methods, it is necessary to carry out complex experiments and data analysis to evaluate the efficiency of soil carbon sink of paddy fields, which requires a lot of manpower and material resources. There are many models for evaluating the efficiency of the soil carbon sink, mainly including a CENTURY model, a RothC model, a DNDC model, a CEVSA model and an EPIC model. Most of these models take into account the influence factors such as meteorology, soil and farmland management measures, and estimate the soil carbon sequestration potential by simulating the process of production, decomposition and transformation of Soil Organic Matters (SOMs). However, because of the differences in data sources, soil carbon sequestration factors and mathematical models used in each model, the results estimated by different scholars are quite different.

Therefore, it is urgent to study and develop a method of scientifically and accurately evaluating efficiency of increasing a soil carbon sink under the condition of returning green manure to fields during a fallow period of a paddy field, in order to realize quantitative calculation, monitoring and verification of efficiency of increasing a soil carbon sink of a paddy field under the condition of returning green manure to fields.

### SUMMARY

The present disclosure aims to provide a method of determining efficiency of increasing a soil carbon sink based on green manure returning to fields and a system thereof, thus realizing the quantitative calculation of efficiency of increasing the soil carbon sink of green manure.

In order to achieve the above object, the present disclosure provides the following solution.

A method of determining efficiency of increasing a soil carbon sink based on green manure returning to fields is provided, including:
determining green manure biomass of a target farmland based on an unmanned aerial vehicle lidar technology during a fallow period of the target farmland before green manure is turned over;
calculating carbon biomass of the target farmland based on the green manure biomass;
calculating microbial soil carbon pump efficiency and mineral carbon pump efficiency after the green manure of the target farmland is turned over; and
determining a soil carbon sink effect of turning over green manure on the target farmland according to the carbon biomass, the microbial soil carbon pump efficiency and the mineral carbon pump efficiency.

Preferably, determining green manure biomass of a target farmland based on an unmanned aerial vehicle lidar technology during a fallow period of the target farmland before green manure is turned over specifically includes:
determining a planting area of green manure of the target farmland using unmanned aerial vehicle lidar;
calculating the green manure biomass of the target farmland based on the planting area of green manure.

Preferably, the green manure biomass is calculated in the following formula:

$$A G {B}_{T} = 1 \text{.327} \cdot {A}_{i} \cdot {V}_{i} \cdot {BEF}_{i}^{ln [ ( \frac{{C}_{i n}}{{L}_{i n}} {K}_{a g} ) \cdot t ) ]} \cdot {ρ}_{i} ;$$

where AGB_{T} denotes the green manure biomass, Aᵢ denotes the planting area of an i-th kind of green manure, Vᵢ denotes accumulation of an i-th kind of green manure, BEFᵢ denotes a biomass expansion coefficient of an i-th kind of green manure, Cᵢₙ denotes an absorption amount of carbon dioxide during a growth period of an i-th kind of green manure, Lᵢₙ denotes a light energy absorption amount of an i-th kind of green manure, K_{ag} denotes a consumption rate generated by life activities of green manure, t denotes growth time of green manure, and ρᵢ denotes a growth density of an i-th kind of green manure.

Preferably, the carbon biomass of the target farmland is calculated in the following formula:

$$A G {B}_{T}^{C} = {δ}_{i} \cdot \frac{{\int }_{S} ( α ρ + β ) d ρ}{A G {B}_{a g}} + {ε}_{i} \cdot ( \frac{{\int }_{0}^{D} ( \frac{{m}^{′}}{V} {e}^{λ \cdot {AGB}_{T}} ) dλ}{A G {B}_{b g}} ) + C ;$$

where AGB_{T}^{C} denotes carbon biomass of the target farmland, δᵢ denotes an aboveground carbon biomass coefficient of an i-th kind of green manure, α denotes a first regression coefficient, β denotes a second regression coefficient, εᵢ denotes an underground carbon biomass coefficient of an i-th kind of green manure, C denotes a constant, AGB_{ag} denotes aboveground biomass, AGB_{bg} denotes underground biomass, D denotes a soil depth, m′ denotes a soil quality, V denotes a soil volume, λ denotes a third regression coefficient, AGB_{T} denotes the green manure biomass, S denotes a land area of the target farmland, and ρ denotes a growth density of green manure.

Preferably, the microbial soil carbon pump efficiency is calculated in the following formula:

$$M i P E = {\sum }_{n = 1 , 2 , 3}^{Δ τ , Δ σ , Δ ω} ( {ACP}^{ln \frac{( Δ τ + Δ σ + Δ ω )}{Δ {τ}^{\sqrt{{b}^{2} - {a}^{2}}}}} , SC {P}^{ln \frac{( Δ τ + Δ σ + Δ ω )}{2 Δ {σ}^{\sqrt{{b}^{2} - {a}^{2}}}}} , IC {P}^{ln \frac{( Δ τ + Δ σ + Δ ω )}{3 Δ {ω}^{\sqrt{{b}^{2} - {a}^{2}}}}} ) ;$$

where MiPE denotes the microbial soil carbon pump efficiency, ACP denotes an activated carbon pool, SCP denotes a slow carbon pool, ICP denotes an inert carbon pool, Δτ denotes a total carbon increment of the activated carbon pool, Δσ denotes a total carbon increment of the slow carbon pool, Δω denotes a total carbon increment of the inert carbon pool, a denotes a total soil carbon amount before the green manure returns to fields, and b denotes a total soil carbon amount after the green manure returns to fields.

Preferably, a soil carbon sink effect of turning over green manure on the target farmland is determined according to the carbon biomass, the microbial soil carbon pump efficiency and the mineral carbon pump efficiency in the following formula:

$$C S = ( E x {p}_{i} \cdot MiPE + {Exp}_{j} \cdot MnPE ) \cdot {A}_{i} \cdot \frac{0.58 \cdot {AGB}_{T}^{C} \cdot BD \cdot D \cdot ( 1 - G )}{100} ;$$

where CS denotes a soil carbon sink effect, Exp_{i′} denotes a microbial carbon pump efficiency coefficient, Expⱼ denotes a mineral carbon pump efficiency coefficient, MiPE denotes the microbial soil carbon pump efficiency, MnPE denotes the mineral carbon pump efficiency, AGB_{T}^{C} denotes the carbon biomass of the target farmland, Aᵢ denotes an area returning to fields, BD) denotes a soil bulk density, D denotes a soil depth, and G denotes a percentage of gravel with a diameter of ≥2 mm in volume.

Preferably, the target farmland is a paddy field.

The present disclosure further discloses a system of determining efficiency of increasing a soil carbon sink based on green manure returning to fields, including:
a green manure biomass determining module, which is configured to determine green manure biomass of a target farmland based on an unmanned aerial vehicle lidar technology during a fallow period of the target farmland before green manure is turned over;
a carbon biomass determining module, which is configured to calculate carbon biomass of the target farmland based on the green manure biomass;
a microbial soil carbon pump efficiency and mineral carbon pump efficiency calculating module, which is configured to calculate microbial soil carbon pump efficiency and mineral carbon pump efficiency after the green manure of the target farmland is turned over; and
a soil carbon sink effect determining module, which is configured to determine a soil carbon sink effect of turning over green manure on the target farmland according to the carbon biomass, the microbial soil carbon pump efficiency and the mineral carbon pump efficiency.

According to the specific embodiment provided by the present disclosure, the present disclosure discloses the following technical effects.

According to the carbon biomass, a soil carbon sink effect of turning over green manure on the target farmland is determined according to the carbon biomass, the microbial soil carbon pump efficiency and the mineral carbon pump efficiency. The present disclosure realizes the quantitative calculation of efficiency of increasing the soil carbon sink of green manure, and provides technical support for evaluating and managing a farmland soil organic carbon sink.

### BRIEF DESCRIPTION OF THE DRAWINGS

In order to explain the embodiments of the present disclosure or the technical solutions in the prior art more clearly, the drawings that need to be used in the embodiments will be briefly introduced. Obviously, the drawings in the following description are only some embodiments of the present disclosure. For those skilled in the art, other drawings can be obtained according to these drawings without creative labor.

FIG. 1 is a schematic flow chart of a method of determining efficiency of increasing a soil carbon sink based on green manure returning to fields according to an embodiment of the present disclosure.

FIG. 2 is a schematic structural diagram of a system of determining efficiency of increasing a soil carbon sink based on green manure returning to fields according to an embodiment of the present disclosure.

### DETAILED DESCRIPTION OF THE EMBODIMENTS

The technical solutions in the embodiments of the present disclosure will be clearly and completely described with reference to the drawings in the embodiments of the present disclosure hereinafter. Obviously, the described embodiments are only some embodiments of the present disclosure, rather than all of the embodiments. Based on the embodiment of the present disclosure, all other embodiments obtained by those skilled in the art without creative labor fall within the scope of protection of the present disclosure.

The present disclosure aims to provide a method of determining efficiency of increasing a soil carbon sink based on green manure returning to fields and a system thereof, which realizes the quantitative calculation of efficiency of increasing the soil carbon sink of green manure.

In order to make the above objects, features and advantages of the present disclosure more obvious and understandable, the present disclosure will be explained in further detail with reference to the drawings and detailed description hereinafter.

### Embodiment 1

In this embodiment, first, the planting biomass of green manure in the fallow period is evaluated to obtain information on the total aboveground and underground biomass of green manure; second, the total carbon invested into paddy field soil is calculated; and finally, a soil carbon sink is gradually formed under the action of microbial decomposition of carbon biomass and utilization and mineral protection, the soil carbon holding increment and its efficiency are estimated, and the influence of green manure investment on efficiency of the soil carbon sink is evaluated.

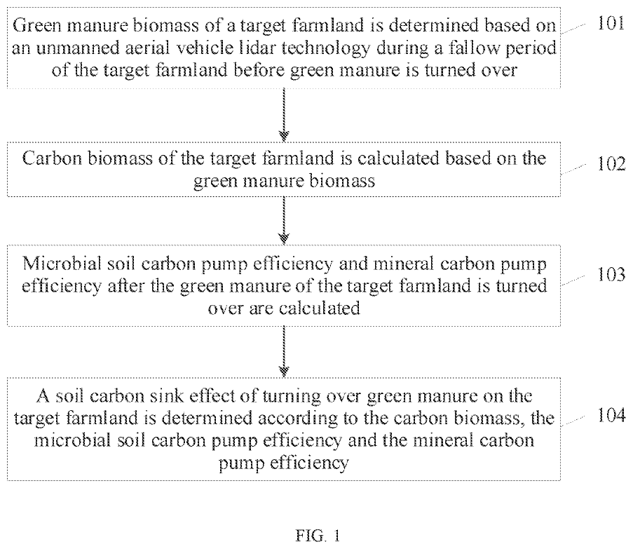

As shown in FIG. 1, this embodiment provides a method of determining efficiency of increasing a soil carbon sink based on green manure returning to fields. The method includes the following steps.

Step 101: green manure biomass of a target farmland is determined based on an unmanned aerial vehicle lidar technology during a fallow period of the target farmland before green manure is turned over.

Step 101 specifically includes:
determining a planting area of green manure of the target farmland using unmanned aerial vehicle lidar;
calculating the green manure biomass of the target farmland based on the planting area of green manure.

The green manure biomass is calculated in the following formula:

$$AG {B}_{T} = 1 \text{.327} \cdot {A}_{i} \cdot {V}_{i} \cdot {BEF}_{i}^{ln [ ( \frac{{C}_{i n}}{{L}_{i n}} {K}_{a g} ) \cdot t ) ]} \cdot {ρ}_{i} ;$$

where AGB_{T} denotes the green manure biomass (kg/m²), Aᵢ denotes the planting area (hm²) of an i-th kind of green manure, Vᵢ denotes accumulation (m³) of an i-th kind of green manure, BEFᵢ denotes a biomass expansion coefficient of an i-th kind of green manure, Cᵢₙ denotes an absorption amount (kg) of carbon dioxide during a growth period of an i-th kind of green manure, Lᵢₙ denotes a light energy absorption amount (lux) of an i-th kind of green manure, K_{ag} denotes a consumption rate (mg(μl)/(h·g)) generated by life activities of green manure, t denotes growth time (h) of green manure, and ρᵢ denotes a growth density (kg/m³) of an i-th kind of green manure.

The i-th kind is the kind of green manure corresponding to the target farmland. The kinds of green manure include Chinese milk vetch, alfalfa and Vicia sativa.
1) The accumulation Vᵢ of green manure is the volume, in which the green manure coverage per unit area of land after green manure is completely crushed is mainly taken into account herein, indicating the uniformity of returning green manure which has been turned over to fields.
2) All of the green manure carbon sequestration comes from absorbing CO₂ in the air. According to the general chemical equilibrium equation of plantphotosynthesis 6CO₂+12H₂O═C₆H₁₂O₆+6H₂O+6O₂, it can be estimated that 264 g of carbon dioxide can be absorbed for every 72 g of sequestrated carbon.
3) The light energy absorption amount can be evaluated by the light energy utilization index of plants. The light energy utilization rate is an index to measure the degree of light energy utilization and the production level of crops in a certain area, that is to say, the ratio of the energy contained in organic matters accumulated by plant photosynthesis in a certain period of time per unit area of land to the amount of solar radiation irradiated on the land in the same period.

Step 102: carbon biomass of the target farmland is calculated based on the green manure biomass.

The carbon biomass of the target farmland is calculated in the following formula:

$$AG {B}_{T}^{C} = {δ}_{i} \cdot \frac{{\int }_{S} ( α ρ + β ) d ρ}{A G {B}_{a g}} + {ε}_{i} \cdot ( \frac{{\int }_{0}^{D} ( \frac{{m}^{′}}{V} {e}^{λ \cdot {AGB}_{T}} ) dλ}{A G {B}_{b g}} ) + C ;$$

where AGB_{T}^{C} denotes carbon biomass of the target farmland, δᵢ denotes an aboveground carbon biomass coefficient of an i-th kind of green manure, α denotes a first regression coefficient, β denotes a second regression coefficient, εᵢ denotes an underground carbon biomass coefficient of an i-th kind of green manure, C denotes a constant, AGB_{ag} denotes aboveground biomass, AGB_{bg} denotes underground biomass,

$${δ}_{i} \cdot \frac{{\int }_{S} ( α ρ + β ) d ρ}{A G {B}_{a g}}$$

denotes an aboveground carbon biomass,

$${ε}_{i} \cdot ( \frac{{\int }_{0}^{D} ( \frac{{m}^{′}}{V} {e}^{λ \cdot {AGB}_{T}} ) dλ}{A G {B}_{b g}} )$$

denotes an underground carbon biomass, D denotes a soil depth, m′ denotes a soil quality, V denotes a soil volume, λ denotes a third regression coefficient, AGB_{T} denotes the green manure biomass, S denotes a land area of the target farmland, and ρ denotes a growth density of green manure.

Step 103: microbial soil carbon pump efficiency and mineral carbon pump efficiency after the green manure of the target farmland is turned over are calculated.

The microbial soil carbon pump efficiency is calculated in the following formula:

$$MiPE = {\sum }_{n = 1 , 2 , 3}^{Δ τ , Δ σ , Δ ω} ( {ACP}^{ln \frac{( Δ τ + Δ σ + Δ ω )}{Δ {τ}^{\sqrt{{b}^{2} - {a}^{2}}}}} , SC {P}^{ln \frac{( Δ τ + Δ σ + Δ ω )}{2 Δ {σ}^{\sqrt{{b}^{2} - {a}^{2}}}}} , IC {P}^{ln \frac{( Δ τ + Δ σ + Δ ω )}{{Δω}^{\sqrt{{b}^{2} - {a}^{2}}}}} ) ;$$

where MiPE denotes the microbial soil carbon pump efficiency, ACP denotes an activated carbon pool, SCP denotes a slow carbon pool, ICP denotes an inert carbon pool, Δτ denotes a total carbon increment (Mg/m²) of the activated carbon pool, Δσ denotes a total carbon increment (Mg/m²) of the slow carbon pool, Δω denotes a total carbon increment (Mg/m²) of the inert carbon pool, a denotes a total soil carbon amount (Mg/m²) before the green manure returns to fields, and b denotes a total soil carbon amount (Mg/m²) after the green manure returns to fields.

The mineral carbon pump efficiency is calculated in the following formula:

$$M n P E = {\sum }_{n = 1 , 2 , 3}^{Δ τ , Δ σ , Δ ω} ( 0.3 \cdot {ACP}^{ln \frac{( Δ τ + Δ σ + Δ ω )}{Δ {τ}^{\sqrt{{b}^{2} - {a}^{2}}}}} , 0.2 \cdot {SCP}^{ln \frac{( Δ τ + Δ σ + Δ ω )}{2 Δ {σ}^{\sqrt{{b}^{2} - {a}^{2}}}}} , 0.1 \cdot IC {P}^{ln \frac{( Δ τ + Δ σ + Δ ω )}{{Δω}^{\sqrt{{b}^{2} - {a}^{2}}}}} )$$

where MnPE denote the mineral carbon pump efficiency.

Step 104: a soil carbon sink effect of turning over green manure on the target farmland is determined according to the carbon biomass, the microbial soil carbon pump efficiency and the mineral carbon pump efficiency.

A soil carbon sink effect of turning over green manure on the target farmland is determined in the following formula:

$$C S = ( E x {p}_{i} \cdot MiPE + {Exp}_{j} \cdot MnPE ) \cdot {A}_{i} \cdot \frac{0.58 \cdot {AGB}_{T}^{C} \cdot BD \cdot D \cdot ( 1 - G )}{100} ;$$

where CS denotes a soil carbon sink effect, that is, the number of carbon sinks (Pg C·yr−1), Exp_{i′} denotes a microbial carbon pump efficiency coefficient, Expⱼ denotes a mineral carbon pump efficiency coefficient, MiPE denotes the microbial soil carbon pump efficiency, MnPE denotes the mineral carbon pump efficiency, n is a set constant, AGB_{T}^{C} denotes the carbon biomass of the target farmland, Aᵢ denotes an area returning to fields, that is, the planting area (hm²) of an i-th kind of green manure, BD denotes a soil bulk density (kg/m³), D denotes a soil depth (m), and G denotes a percentage of gravel with a diameter of ≥2 mm in volume.

The present disclosure provides a method of determining efficiency of increasing a soil carbon sink based on green manure returning to fields, which is to evaluate the relationship between the biomass and the soil carbon sink under the condition of turning over green manure on the basis of obtaining green manure aboveground biomass by the unmanned aerial vehicle lidar. The present disclosure fills the technical gap related to the evaluation of efficiency of increasing a soil carbon sink by returning green manure to fields, provides a method basis for efficient and rapid evaluation of the soil carbon sink under the condition of returning green manure to fields in a regional paddy field, and provides convenience for quantitative calculation, efficiency evaluation, carbon monitoring and carbon verification of efficiency of the soil carbon sink in a paddy field.

### Embodiment 2

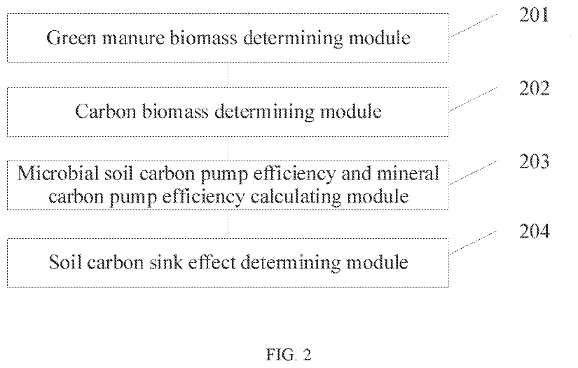

As shown in FIG. 2, this embodiment provides a system of determining efficiency of increasing a soil carbon sink based on green manure returning to fields. The system includes:
a green manure biomass determining module 201, which is configured to determine green manure biomass of a target farmland based on an unmanned aerial vehicle lidar technology during a fallow period of the target farmland before green manure is turned over;
a carbon biomass determining module 202, which is configured to calculate carbon biomass of the target farmland based on the green manure biomass;
a microbial soil carbon pump efficiency and mineral carbon pump efficiency calculating module 203, which is configured to calculate microbial soil carbon pump efficiency and mineral carbon pump efficiency after the green manure of the target farmland is turned over; and
a soil carbon sink effect determining module 204, which is configured to determine a soil carbon sink effect of turning over green manure on the target farmland according to the carbon biomass, the microbial soil carbon pump efficiency and the mineral carbon pump efficiency.

In this specification, various embodiments are described in a progressive way. The differences between each embodiment and other embodiments are highlighted, and the same and similar parts of various embodiments can be referred to each other. Since the system disclosed in the embodiment corresponds to the method disclosed in the embodiment, the system is described simply. Refer to the description of the method of the relevant points.

In the present disclosure, specific examples are applied to illustrate the principle and implementation of the present disclosure, and the explanations of the above embodiments are only used to help understand the method and core ideas of the present disclosure. At the same time, according to the idea of the present disclosure, there will be some changes in the specific implementation and application scope for those skilled in the art. To sum up, the contents of the specification should not be construed as limiting the present disclosure.

What is claimed is:

1. A method of determining efficiency of increasing a soil carbon sink based on green manure returning to fields, comprising:
determining green manure biomass of a target farmland based on an unmanned aerial vehicle lidar technology during a fallow period of the target farmland before green manure is turned over;
calculating carbon biomass of the target farmland based on the green manure biomass;
calculating microbial soil carbon pump efficiency and mineral carbon pump efficiency after the green manure of the target farmland is turned over; and
determining a soil carbon sink effect of turning over green manure on the target farmland according to the carbon biomass, the microbial soil carbon pump efficiency and the mineral carbon pump efficiency.

2. The method of determining efficiency of increasing the soil carbon sink based on green manure returning to fields according to claim 1, wherein determining green manure biomass of a target farmland based on an unmanned aerial vehicle lidar technology during a fallow period of the target farmland before green manure is turned over specifically comprises:
determining a planting area of green manure of the target farmland using unmanned aerial vehicle lidar;
calculating the green manure biomass of the target farmland based on the planting area of green manure.

3. The method of determining efficiency of increasing the soil carbon sink based on green manure returning to fields according to claim 2, wherein the green manure biomass is calculated in the following formula:
$$AG {B}_{T} = 1 \text{.327} \cdot {A}_{i} \cdot {V}_{i} \cdot {BEF}_{i}^{ln [ ( \frac{{C}_{i n}}{{L}_{i n}} {K}_{a g} ) \cdot t ) ]} \cdot {ρ}_{i} ;$$
where AGB_{T} denotes the green manure biomass, Aᵢ denotes the planting area of an i-th kind of green manure, Vᵢ denotes accumulation of an i-th kind of green manure, BEFᵢ denotes a biomass expansion coefficient of an i-th kind of green manure, Cᵢₙ denotes an absorption amount of carbon dioxide during a growth period of an i-th kind of green manure, Lᵢₙ denotes a light energy absorption amount of an i-th kind of green manure, K_{ag} denotes a consumption rate generated by life activities of green manure, t denotes growth time of green manure, and ρᵢ denotes a growth density of an i-th kind of green manure.

4. The method of determining efficiency of increasing a soil carbon sink based on green manure returning to fields according to claim 1, wherein the carbon biomass of the target farmland is calculated in the following formula:
$$AG {B}_{T}^{C} = {δ}_{i} \cdot \frac{{\int }_{S} ( α ρ + β ) d ρ}{A G {B}_{a g}} + {ε}_{i} \cdot ( \frac{{\int }_{0}^{D} ( \frac{{m}^{′}}{V} {e}^{λ \cdot {AGB}_{T}} ) dλ}{A G {B}_{b g}} ) + C ;$$
where AGB_{T}^{C} denotes carbon biomass of the target farmland, δᵢ denotes an aboveground carbon biomass coefficient of an i-th kind of green manure, α denotes a first regression coefficient, β denotes a second regression coefficient, εᵢ denotes an underground carbon biomass coefficient of an i-th kind of green manure, C denotes a constant, AGB_{ag} denotes aboveground biomass, AGB_{bg} denotes underground biomass, D denotes a soil depth, m′ denotes a soil quality, V denotes a soil volume, λ denotes a third regression coefficient, AGB_{T} denotes the green manure biomass, S denotes a land area of the target farmland, and ρ denotes a growth density of green manure.

5. The method of determining efficiency of increasing the soil carbon sink based on green manure returning to fields according to claim 1, wherein the microbial soil carbon pump efficiency is calculated in the following formula:
$$MiPE = {\sum }_{n = 1 , 2 , 3}^{Δ τ , Δ σ , Δ ω} ( {ACP}^{ln \frac{( Δ τ + Δ σ + Δ ω )}{Δ {τ}^{\sqrt{{b}^{2} - {a}^{2}}}}} , SC {P}^{ln \frac{( Δ τ + Δ σ + Δ ω )}{2 Δ {σ}^{\sqrt{{b}^{2} - {a}^{2}}}}} , IC {P}^{ln \frac{( Δ τ + Δ σ + Δ ω )}{{Δω}^{\sqrt{{b}^{2} - {a}^{2}}}}} ) ;$$
where MiPE denotes the microbial soil carbon pump efficiency, ACP denotes an activated carbon pool, SCP denotes a slow carbon pool, ICP denotes an inert carbon pool, Δτ denotes a total carbon increment of the activated carbon pool, Δσ denotes a total carbon increment of the slow carbon pool, Δω denotes a total carbon increment of the inert carbon pool, a denotes a total soil carbon amount before the green manure returns to fields, and b denotes a total soil carbon amount after the green manure returns to fields.

6. The method of determining efficiency of increasing the soil carbon sink based on green manure returning to fields according to claim 1, wherein a soil carbon sink effect of turning over green manure on the target farmland is determined according to the carbon biomass, the microbial soil carbon pump efficiency and the mineral carbon pump efficiency in the following formula:
$$C S = ( E x {p}_{i} \cdot MiPE + {Exp}_{j} \cdot MnPE ) \cdot {A}_{i} \cdot \frac{0.58 \cdot {AGB}_{T}^{C} \cdot BD \cdot D \cdot ( 1 - G )}{100} ;$$
where CS denotes a soil carbon sink effect, Exp_{i′} denotes a microbial carbon pump efficiency coefficient, Expⱼ denotes a mineral carbon pump efficiency coefficient, MiPE denotes the microbial soil carbon pump efficiency, MnPE denotes the mineral carbon pump efficiency, AGB_{T}^{C} denotes the carbon biomass of the target farmland, Aᵢ denotes an area returning to fields, BD denotes a soil bulk density, D denotes a soil depth, and G denotes a percentage of gravel with a diameter of ≥2 mm in volume.

7. The method of determining efficiency of increasing the soil carbon sink based on green manure returning to fields according to claim 1, wherein the target farmland is a paddy field.

8. A system of determining efficiency of increasing a soil carbon sink based on green manure returning to fields, comprising:
a green manure biomass determining module, which is configured to determine green manure biomass of a target farmland based on an unmanned aerial vehicle lidar technology during a fallow period of the target farmland before green manure is turned over;
a carbon biomass determining module, which is configured to calculate carbon biomass of the target farmland based on the green manure biomass;
a microbial soil carbon pump efficiency and mineral carbon pump efficiency calculating module, which is configured to calculate microbial soil carbon pump efficiency and mineral carbon pump efficiency after the green manure of the target farmland is turned over; and
a soil carbon sink effect determining module, which is configured to determine a soil carbon sink effect of turning over green manure on the target farmland according to the carbon biomass, the microbial soil carbon pump efficiency and the mineral carbon pump efficiency.
